(19) United States
(12) Patent Application Publication
(10) Pub. No.: US 2007/0078450 A1
(43) Pub. Date: Apr. 5, 2007

(54) Method and apparatus for characterization of tissue using catheter-based spectroscopy

(76) Inventor: Mark G. Langer, Mequon, WI (US)

Correspondence Address: JOSEPH S. HEINO, ESQ., 111 E. KILBOURN AVENUE, SUITE 1400, MILWAUKEE, WI, 53202, US

(21) Appl. No.: 11/240,992

(22) Filed: Sep. 30, 2005

(51) Int. Cl. A61B 18/18

(52) U.S. Cl. 606/12

(57) ABSTRACT

A tissue characterization apparatus and method may comprise a light source configured to direct light towards the tissue, a light receptor configured to receive light reflected from the tissue and configured to generate an electrical signal, and a spectrogram configured to analyze the signal generated by the light receptor.

### FIELD

The present invention is related to a method and apparatus for treating atrial fibrillation. More specifically, the present invention relates to a method and apparatus for characterizing ablated tissue using spectroscopy.

### BACKGROUND

A normal human heart includes a right ventricle, a right atrium, a left ventricle and a left atrium. The right atrium is in fluid communication with the superior vena cava and the inferior vena cava. The atrioventricular septum separates the right atrium from the right ventricle. The tricuspid valve contained within the atrioventricular septum communicates the right atrium with the right ventricle.

In the normal heart, contraction and relaxation of the heart muscle (myocardium) takes place in an organized fashion as electro-chemical signals pass sequentially through the myocardium from the sinoatrial (SA) node to the atrioventricular (AV) node and then along a well defined path which includes the His-Purkinge system into the left and right ventricles. Initial electrical impulses are generated at the SA node and conducted to the AV node. The AV node lies near the ostium of the coronary sinus in the interatrial septum in the right atrium. The His-Purkinje system begins at the AV node and follows along the membranous interatrial septum toward the tricuspid valve, through the atrioventricular septum and into the membranous interventricular septum. At approximately the middle of the interventricular septum, the His-Purkinje system splits into the right and left branches of the interventricular septum.

Abnormal rhythms in the heart are generally referred to as arrhythmias. Arrhythmias that occur in the atria are normally referred to as atrial arrhythmias and can result in significant patient discomfort and even death due to the following: an irregular heart rate, which can cause patient discomfort and anxiety; loss of synchronous atrioventricular contractions which compromises cardiac hemodynamics resulting in varying levels of congestive heart failure; and stasis of blood flow, which increases the likelihood of thromboembolism, among other things.

Atrial fibrillation is a common heart rhythm disturbance. It affects millions of patients per year in the United States alone. It is benign arrhythmia, but there is a lack of effective and safe therapies. Atrial fibrillation is a rapid and highly irregular heart arrhythmia caused by chaotic electrical impulses in the atria of the heart. During atrial fibrillation, the AV node and the ventricles are bombarded by these irregular electrical impulses. As a result, the heart rate becomes fast and irregular and the normal coordination between the atria and the ventricles is lost.

Among the dangers of atrial fibrillation is that, if the arrhythmia is sustained, the ineffective pumping action of the atria can allow blood clots to form within the heart. If the blood clots break off and get into the bloodstream, a stroke can result. Atrial fibrillation can also cause palpitations, easy fatigability, shortness of breath and lightheadedness.

Currently, several methods are used to restore a normal heart rhythm, among them being ablation, antiarrhythmic drugs, and cardioversion. Antiarrhythmic drugs can restore a normal rhythm and can have side effects. Cardioversion involves placing the patient in an anesthesia-induced sleep and administering and electrical charge to the patient's chest.

A relatively recent innovation is the development of an implantable atrial defibrillator. An atrial defibrillator is a device that detects atrial fibrillation when it occurs and administers a small shock in order to restore a normal heart rhythm.

Another approach to treating atrial fibrillation has been to create a series of surgical incisions through the atrial myocardium in a preselected pattern so as to create conductive corridors of viable tissue bounded by scar tissue.

Yet another approach to treating arrhythmia is transmural ablation of the arrhythmia itself. During a typical ablation procedure, an electrophysiology study of the heart is performed, that is, the electrical system of the heart is mapped, a troublesome area is identified and that area is ablated. Such a procedure may either be performed from within the chambers of the heart using an endovascular device (e.g., catheters) introduced through arteries or veins or from outside the heart using devices introduced through the chest. Ablation involves placing an energy source in contact with cardiac tissue to heat the tissue and create a permanent scar or lesion. Various ablation techniques can be utilized including the use of cryogenic, radiofrequency (RF), laser and microwave. The lesions are designed to interrupt existing conduction pathways commonly associated with arrhythmias within the heart.

In order to ablate tissue, energy is applied via an energy source to create a lesion without regard to the specific level of electromagnetic energy supplied by the generator. In situations where too much energy is applied, a crater can be formed at the contact site. Normally, the crater is formed by cells within the tissue actually exploding and may cause a lesion that is much larger than desired.

Accordingly, it would be advantageous to provide a method and apparatus for determining whether tissue has been sufficiently ablated such that it no longer conducts electricity. It would also be advantageous to provide a method and apparatus for the prevention of crater formation during ablation procedures. It would further be advantageous to provide a method and apparatus for providing real time tissue differentiation for determining whether a specific sample of tissue is sufficiently ablated during catheter-based procedures.

### SUMMARY

One embodiment of the present invention relates to a tissue characterization apparatus comprising a light source configured to direct light towards the tissue, a light receptor configured to receive light reflected from the tissue and configured to generate an electrical signal, and a spectrogram configured to analyze the signal generated by the light receptor.

Another embodiment of the present invention relates to a tissue characterization apparatus comprising a catheter having a distal end and a proximal end, a Near Infra-Red (NIR) light source configured to direct light towards the tissue, a light receptor configured to receive light reflected from the tissue and generate an electrical signal, and a spectrogram configured to analyze the signal generated by the light receptor.

Another embodiment of the present invention relates to a method of ablating tissue comprising the steps of utilizing a catheter comprising an ablation electrode to ablate tissue, directing (NIR) energy toward the tissue that is ablated, collecting (NIR) energy that is reflected from the tissue that is ablated, and generating data corresponding to the reflected (NIR) energy.

### BRIEF DESCRIPTION OF THE DRAWINGS

FIG. 1 is a schematic block diagram of an ablation system according to an exemplary embodiment.

FIG. 2 is a schematic representation of an ablation apparatus according to an exemplary embodiment.

FIG. 3 is a cross-sectional diagram of fibers of an ablation apparatus according to an exemplary embodiment.

FIG. 4 is a flow diagram of a process for characterizing tissue according to an exemplary embodiment.

### DETAILED DESCRIPTION

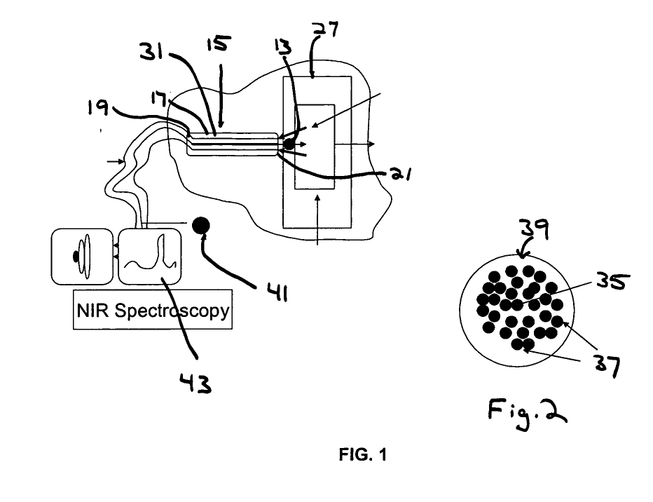

Referring to FIG. 1, a block diagram of an ablation system according to an exemplary embodiment is shown. As shown in FIG. 1, ablation system 10 comprises ablation controller circuitry 3, an ablation energy generator 5 in electronic communication with ablation controller circuitry 3, and an ablation electrode 7 in electronic communication with ablation energy generator 5.

According to an exemplary embodiment, ablation energy generator 5 provides energy and directs the energy to ablation electrode 7 to ablate tissue. According to various exemplary embodiments, any suitable generator may be used such as DC energy pulse from a defibrillator, RF transmitter, microwave transmitter, laser light source, etc. According to various exemplary embodiments, any suitable type of energy may be used for ablating tissue.

According to an exemplary embodiment, ablation system 10 comprises a light source 41 and a light receptor 33 in communication with controller circuitry 3. Light source 41, which directs (NIR) energy towards the heart tissue and the light receptors 33, is operable to detect the incidence of light reflected from tissue.

Ablation system 10 may also include cardiac sensing circuitry 11. According to an exemplary embodiment, cardiac sensing circuitry 11 may, for example, detect atrial and/or ventricular activity and may provide an ECG waveform signal representative of cardiac activity. According to various exemplary embodiments, the cardiac sensing circuitry may include any circuitry known in the art for detecting cardiac activity.

As shown in FIG. 2, ablation electrode 13 attaches to and/or forms a part of a catheter 15 or another medical device configured to be positioned within the heart. According to various exemplary embodiments, the ablation electrode may comprise any suitable electrode.

According to an exemplary embodiment, ablation catheter 15 as shown in FIG. 2 comprises a catheter body 17 having a proximal end 19, a distal end 21 and at least one lumen 23 extending substantially through the catheter body 17, a plurality of openings 25 in the surface of the catheter 15 extending through the outside surface of the catheter into lumen 23, and a system for introduction of a conductive media into lumen 23, whereby the conductive media passes through lumen 23 and is expelled out one or more openings 25 in catheter 15 to contact tissue 27 to be ablated. According to various exemplary embodiments, any suitable system for controlling the flow of the conductive media through the lumen to create a consistent flow of conductive media out through the one or more openings in the catheter may be used.

According to an exemplary embodiment, catheter body 15 comprises an elongated catheter made of materials suitable for use in humans, such as nonconductive polymers. According to a particularly preferred embodiment, the catheter is between 0.5 meters and 1.5 meters in length and between 1⅓ mm and 5 mm in diameter. According to a particularly preferred embodiment, the catheter contains one or more lumens sufficient to accommodate wires for one or more sensing electrodes, such as thermosensing devices. According to an exemplary embodiment, catheter body 17 comprises a system for controlling movement of the catheter 15 such as a pull wire (not shown). According to various exemplary embodiments, any suitable catheter may be used.

Referring to FIG. 2, (NIR) energy is projected through a fiber optic channel 31 provided in catheter 15 (e.g., a cardiac catheter) to characterize tissue at distal end 21 of catheter 15. According to an exemplary embodiment, catheter 15 is configured to use (NIR) spectroscopy to provide real-time spectrograms displaying tissue properties. Depending on the characteristics of the heart tissue, the tissue absorbs some of the light and reflects some of the light back toward catheter 15. The light reflected back toward catheter 15 is received by light receiving receptors 33 attached to catheter 15.

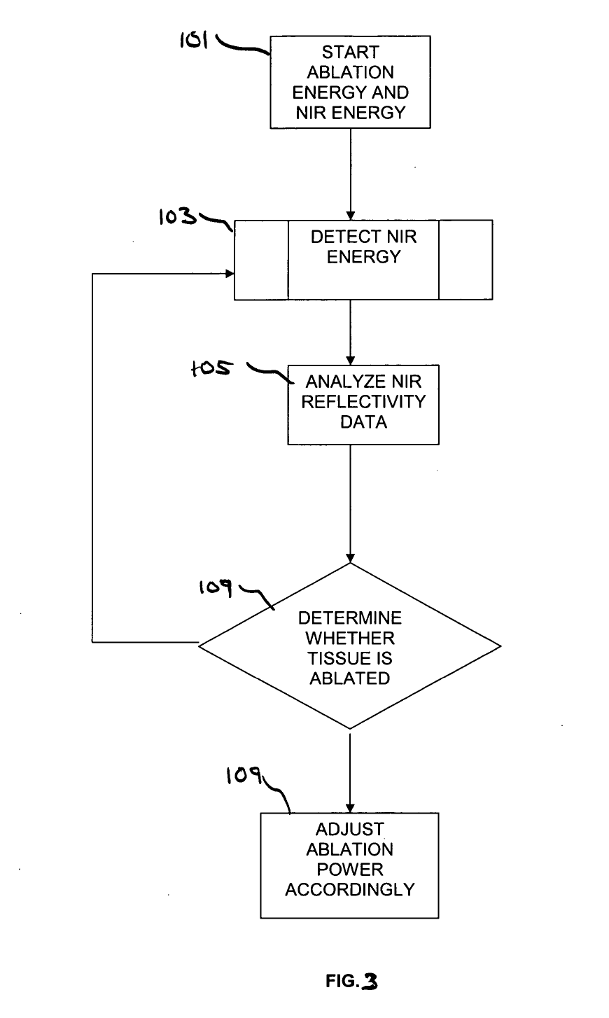

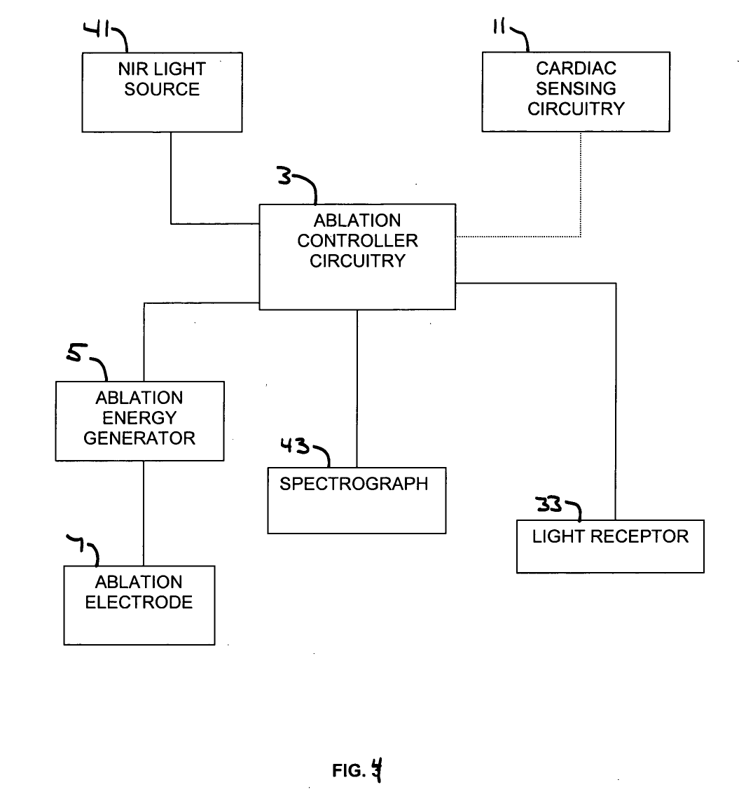

Referring to FIGS. 2-3, catheter 15 comprises a light emitting fiber 35. According to an exemplary embodiment, catheter 15 comprises a plurality of light receiving fibers 37 surrounding the light emitting fiber 35 in a bundle of fibers 39 as shown in FIG. 3. According to an exemplary embodiment, light emitting fiber 35 comprises a remote light source 41 as shown in FIG. 2.

FIG. 2 shows a display 45 of system 10, which is illustrated separate from catheter 15 and may be located in a convenient and suitable viewing area for the physician or clinician performing an ablation procedure. According to an exemplary embodiment, spectrometer 43 is in electrical communication with light receiving. fibers 37. Light reflected from the tissue strikes a photodiode (not shown), which converts the light energy to data or an electrical signal so that the light may be processed by spectrometer 43. The electrical signal is then amplified and displayed on spectrograph 43. FIG. 3 shows a cross-sectional view of light emitting fiber 35 and light receiving fibers 37 according to an exemplary embodiment.

According to various exemplary embodiments, data collection is nearly instantaneous, the (NIR) spectrogram has a high signal to noise ratio, good resolution, is small enough in physical size that it can be inserted into the body via a catheter, etc. According to an exemplary embodiment, the (NIR) spectrometer has resolution <30 m and a speed of between 5-10 ms per scan. The range of the spectrometer is between 1-2.5 μm. According to an exemplary embodiment, the spectrometer signal can be transmitted wirelessly outside of the body to an adjacent spectrograph or alternatively from the distal end of the catheter through fiber optic cable or other suitable wire to the spectrogram outside of the body. In the embodiment shown, the (NIR) energy is passed through fiber optic channel 31 to distal end 21 of catheter 15 into the heart tissue. According to various exemplary embodiments, the (NIR) energy light source may be emitted via semiconductor, tungsten-halogen, etc. As shown in FIG. 2, light source 41 is relayed via fiber optic cable 35 and directed towards heart tissue 27. Some light is absorbed by heart tissue 27 and some light is reflected back towards the light receiving fibers 37. According to an exemplary embodiment, this reflected light is absorbed by light receiving fibers 37 and transmitted back to spectrometer 43 via a fiber optic connection fiber 38, where it is directed to a single element Indium Gallium Arsenide (InGaAs) detector. According to an exemplary embodiment, the InGaAs detector comprises a photodiode that converts the light into electrical signals, which are then amplified by a transimpedance amplifier for processing.

Referring back to FIG. 1, the signal received is then processed by controller circuitry 3. Controller circuitry 3 is operable to recognize various input signals and various sensed signals as well as being operable to filter and display the signals on a spectrograph. According to various exemplary embodiments, the controller circuitry may be operable to perform various suitable processing and analysis functions such as Fourrier analysis, etc. to determine levels of ablation of the affected tissue.

FIG. 4 is a flow chart showing a process 100 for ablating and characterizing tissue according to an exemplary embodiment. In a cardiac ablation procedure, ablation is commenced. As ablation begins, a light emitting fiber directs (NIR) energy towards the heart tissue being ablated. The (NIR) energy is partially absorbed by the heart tissue and partially reflected towards the ablation catheter (step 101). Light receptors receive the reflected energy and the light is conveyed to a photodiode via a light receiving fiber where (NIR) energy is detected (step 103). The photodiode converts said light into an electrical signal, which may be amplified, filtered and/or processed such that an output can be analyzed and/or displayed on a spectrograph 43 (step 105). Based on the data provided by the spectrograph, a determination may be made whether the tissue is sufficiently (e.g., fully) ablated (step 107). Once a determination has been made, the ablation power may be adjusted (e.g., automatically, manually, etc.) according to a desired ablation level (step 109). According to an exemplary embodiment, the method of the present invention may include an algorithm for identifying ablated tissue and reducing ablation energy.

According to various exemplary embodiments, the method may additionally comprise the steps of displaying an automated message to the clinician regarding the level of tissue ablation, and/or reducing power to the ablation electrode if the tissue ablation data reaches predefined criteria (e.g., indications of full ablation).

The construction and arrangement of the elements of the system as shown in the detailed description is illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, orientations, etc.) without materially departing from the novel teachings and advantages of the subject matter recited. For example, elements shown as integrally formed may be constructed of multiple parts or elements shown as multiple parts may be integrally formed, the operation of fasteners, connectors, etc. may be reversed or otherwise varied, etc. The order or sequence of any process or method steps may be varied or re-sequenced according to alternative embodiments. In the claims, any means-plus-function clause is intended to cover the structures described herein as performing the recited function and not only structural equivalents but also equivalent structures. Other substitutions, modifications, changes and omissions may be made in the design, operating conditions and arrangement of the preferred and other exemplary embodiments without departing from the spirit of the present inventions.

What is claimed is:

1. A tissue characterization apparatus comprising:
a light source configured to direct light towards the tissue;
a light receptor configured to receive light reflected from the tissue and configured to generate an electrical signal; and
a spectrogram configured to analyze the signal generated by the light receptor.

2. The tissue characterization device of claim 1, further comprising a catheter.

3. The tissue characterization device of claim 2, wherein the light source and light reflector are provided on a distal end of the catheter.

4. The tissue characterization device of claim 3, wherein the light source comprises a Near Infrared (NIR) light source.

5. The tissue characterization device of claim 4, wherein the spectrogram is configured to display various tissue properties.

6. A tissue characterization apparatus comprising:
a catheter having a distal end and a proximal end;
a Near Infra-Red (NIR) light source configured to direct light towards the tissue;
a light receptor configured to receive light reflected from the tissue and generate an electrical signal; and
a spectrogram configured to analyze the signal generated by the light receptor.

7. The apparatus of claim 6, wherein the (NIR) light source is provided on the distal end of the catheter.

8. The apparatus of claim 6, wherein the catheter comprises an energy source provided at the distal end configured for cardiac ablation.

9. The apparatus of claim 6, wherein the spectrogram is in electronic communication with the light receptor.

10. The apparatus of claim 6, wherein the light receptor is provided on the distal end of the catheter.

11. The apparatus of claim 6, wherein the catheter comprises an energy source for cardiac ablation.

12. The apparatus of claim 11, wherein the energy source is provided at the distal end of the catheter.

13. A method of ablating tissue comprising the steps of:
utilizing a catheter comprising an ablation electrode to ablate tissue;
directing (NIR) energy toward the tissue that is ablated;
collecting (NIR) energy that is reflected from the tissue that is ablated; and
generating data corresponding to the reflected (NIR) energy.

14. The method of claim 13, further comprising the step of displaying the data on a spectrograph.

15. The method of claim 14, wherein the data represents various tissue properties.

16. The method of claim 13, further comprising the step of applying an algorithm to determine whether the tissue is ablated according to predefined criteria.

17. The method of claim 16, wherein the predefined criteria provides an indication regarding the level of ablation that has occurred.

18. The method of claim 17, further comprising the step of reducing power to the ablation electrode if the tissue has been fully ablated.

19. The method of claim 16, further comprising the step of providing an indication that the tissue is ablated according to the predefined criteria.

20. The method of claim 13, further comprising the step of generating an electrical signal corresponding to the (NIR) energy that is reflected from the tissue that is ablated.
